(19) United States
(12) Patent Application Publication
(10) Pub. No.: US 2005/0039240 A1
(43) Pub. Date: Feb. 24, 2005

(54) Multi-usage eyewear supportable on a cap

(76) Inventor: Armand Kidouchim, Woodland Hills, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/644,631

(22) Filed: Aug. 19, 2003

(51) Int. Cl. A61F 9/00

(52) U.S. Cl. 2/10

(57) ABSTRACT

A device associated with a cap, visor or hat having a bill, which allows a wearer to selectively view objects through a lens or face shield includes a mounting member adapted to be removably attached to the bill of the cap. The mounting member may be a sleeve adapted to removably receive the bill, a clip for removable attachment with the bill, or a shell adapted to overlie at least a portion of an exterior surface of the cap and bill. An eye shield is attached to the mounting member so as to be selectively positionable relative to the eyes of the wearer. The eye shield, which includes the lens, is preferably attached to the mounting member such that it can be moved between various positions relative to the eyes of the wearer. The eye shield and lens are also preferably removably attached to the mounting member to accommodate different lenses.

### BACKGROUND OF THE INVENTION

The present invention generally relates to face shields and eyewear. More particularly, the present invention resides in a multi-usage eyewear device or face shield which is supportable on a cap or the like. The present invention is intended to be very flexible so as to enable its incorporation into various industries, as well as for personal use.

The use of vision correcting eyeglasses and sunglasses is well-known. Such glasses are supported on a bridge of the nose of the wearer, and at the temples and behind the ears. However, some individuals find it uncomfortable to wear such eyeglasses or sunglasses due to the irritation and weight of the glasses on the nose and the pressure exerted on the temples and behind the ears of the individual. For some individuals, reading glasses or bifocals are necessary, but must be removed when viewing objects at a distance which is not in focus by such glasses. It is also common for wearers of sunglasses to have to remove the sunglasses when entering a building, a tunnel, etc. which does not have direct exposure to the sunlight and which is too dark with the sunglasses over the individual's eyes.

In some instances, the normal wear of corrective lenses or sunglasses is not feasible. For example, many people who have recently had surgery on the head, such as for cosmetic surgery, are quite sensitive to any contact with objects in areas proximate to the surgical procedure. These persons, however, may still require the use of corrective lenses, but such cannot be supported by the nose, temples or ears. Thus, standard eyeglass frames are out of the question for such people in dealing with their need to correct eyesight.

Aside from personal use, face shields and eyeglasses or lenses are used for various purposes. For example, jewelers often wear magnifying lenses or face shields so as to view intricate detail of the jewelry and avoid eye injury. Carpenters and machine shop employees often wear face shields and sometimes vision correcting lenses or shaded lenses while operating grinders and the like. Of course, dentists and doctors wear such face shields and magnifying lenses for both protection and increased vision, such as when conducting surgery.

In the industrial setting, such lenses and face shields are often incorporated into a headband or hat. Such a headband or hat must be properly adjusted for tight fit to the particular individual wearing the device. Some of these devices can be rather complicated and a tight fit can be difficult to achieve. In others, the devices have been found to be generally uncomfortable for prolonged periods of wearing. Other problems with such prior art headpieces is that they must often be completely removed to remove the lens from alignment of the user's eyes or face. Also, some of these devices, particularly in the medical setting, are intended for single usage only and thus are disposable. Especially when incorporating a magnifying lens, such devices can be rather expensive.

Accordingly, there is a continuing need for a facial shield/eyewear device which is ergonomic and comfortable. Such a device should provide the wearer with panoramic vision. Furthermore, such a device should be capable of incorporating different lenses as needed, usable repetitively and possibly for more than one particular purpose, and be universally usable by virtually all conceivable users. Such a device should also enable the wearer thereof to reposition the lens in an easy manner. The present invention fulfills theses needs and provides other related advantages.

### SUMMARY OF THE INVENTION

The present invention resides in a multi-usage eyewear device which is supportable on a cap or the like.

In a first embodiment, the device, which allows a wearer to selectively view objects through a lens, is associated with a cap having a bill. A mounting member is adapted to be removably attached to the bill of the cap. The mounting member may comprise a sleeve adapted to removably receive a bill of the cap therein or a clip for removable attachment with the bill of the cap. Such a clip could comprise a plurality of elongated arms, with at least one arm being positionable above the bill and at least one arm below the bill so as to retain the bill between the upper and lower arms.

An eye shield, including the lens, is pivotally attached to the mounting member so as to be selectively positionable relative to the eyes of the wearer. In a particularly preferred embodiment, the eye shield is attached to the mounting member by means of a hinge assembly. The hinge assembly comprises a base attached to the mounting member, and articulating body attached at a first end thereof to the base and a second end thereof to an eye shield connector. Preferably, the articulating body is removably attached to the base and the connector is removable attached to the articulating body such that different lenses can be used, or the lens completely removed from the device.

In a second embodiment, the eye shield is removably attached to the bill of a visor. Preferably, the eye shield is removably connected to a connector which is similarly connected to a base attached to the bill of the visor. The articulating body may be incorporated between the connector and the base to allow the eye shield and lens to be selectively positioned relative to the eyes of the wearer.

In another embodiment, the device comprises a face shield attached to the mounting member. The mounting member may comprise a sleeve adapted to removably receive a bill of the cap therein, or a shell adapted to overlie at least a portion of the exterior surface of the cap. A lense is attached to the face shield so as to be positionable relative to the eyes of the wearer. Preferably, the lens is removably attached to the face shield. In a particularly preferred embodiment, the face shield includes a series of generally aligned apertures which removably receive mating protrusions of the lens by snap-fit connections such that the lens is positionable along at least a length of the face shield.

In any of the foregoing embodiments, the lens may comprise a vision correction lens, such as a magnifying lens, or an eye shielding lens, such as a sun shield or the like.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a perspective view of a mounting member in the form of a sleeve which removably receives a bill of a cap, shown in phantom, and a eye shield extending downwardly therefrom;

FIG. 2 is an exploded perspective view of an eye shield and hinge assembly used in accordance with the present invention;

FIG. 3 is a perspective view of the lens and hinge assembly connected to one another;

FIG. 4 is a perspective view of the lens and hinge assembly with the lens pivoted into a different position using the hinge assembly;

FIG. 5 is a perspective view of a sleeve used in accordance with the present invention and having a lens pivotally attached thereto;

FIG. 6 is a perspective view similar to FIG. 5;

FIG. 7 is a perspective view of a mounting member in the form of a clip attached to the hinge assembly and lens in accordance with the present invention;

FIG. 8 is a partially fragmented perspective view of the clip, hinge assembly and lens attached to a bill of a cap;

FIG. 9 is a perspective view of a visor having the hinge assembly and lens in accordance with the present invention;

FIG. 10 is a perspective view of a visor having a lens attached to a bill thereof, and overlying a pair of eyeglasses, shown in phantom;

FIG. 11 is a perspective view of a visor having lenses attached to a bill thereof;

FIG. 12 is a cross-sectional viewtaken generally along lines 12-12 of FIG. 9, illustrating a hinge assembly interconnecting a bill and a lens, with the lens being positioned so as to be aligned with eyes of the wearer;

FIG. 13 is a cross-sectional view similar to FIG. 12, with the lens being folded away so as to be out of alignment with eyes of the wearer;

FIG. 14 is a cross-sectional view similar to FIG. 12;

FIG. 15 is a cross-sectional view similar to FIG. 13, but having the lens folded inward instead of outward;

FIG. 16 is a perspective view of another device embodying the present invention including a sleeve overlying a bill of a cap, shown in phantom, and having a face shield extending downwardly therefrom and a lens attached to the face shield;

FIG. 17 is a rear elevational view of the sleeve, face shield and lens of FIG. 16;

FIG. 18 is a partially exploded perspective view of the device of FIGS. 16 and 17;

FIG. 19 is a perspective view of a face shield of the present invention with a lens movably positioned thereon;

FIG. 20 is a cross-sectional view taken generally along line 20-20 of FIG. 18, illustrating a snap-fit connection between the lens and face shield;

FIG. 21 is an enlarged cross-sectional view taken of area “21” of FIG. 20, illustrating a protrusion of the lens inserted into an aperture of the face shield;

FIG. 22 is a perspective view of the face shield and lens attached to a shield adapted to be placed over a cap; and

FIG. 23 is a perspective view of the face shield and lens attached to another shield adapted to be placed over a cap or the like.

### DETAILED DESCRIPTION OF THE PREFERRED EMBODIMENTS

As shown in the accompanying drawings for purposes of illustration, the present invention resides in a device which is associated with a cap, visor, hat or the like having a bill, which allows a wearer to selectively view objects through a lens, such as a vision correction lens, magnifying lens, shading lens, etc.

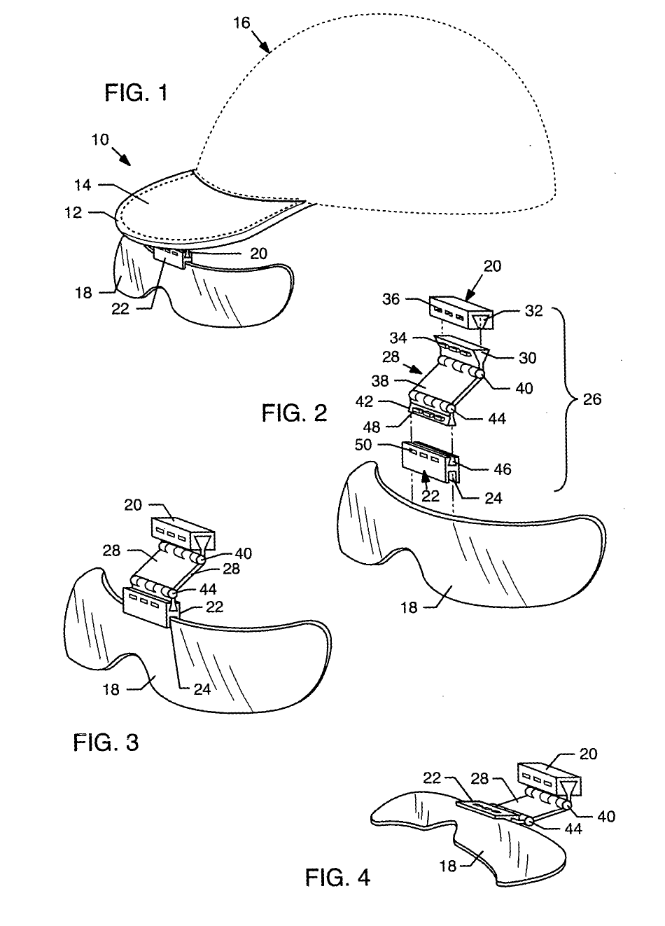

With reference now to FIGS. 1-4, in one embodiment the device 10 is comprised of a mounting member in the form of a sleeve 12 which is configured to removably receive and be placed upon a bill 14 of a cap 16 or other hat having such a bill 14. One of the premises of the present invention is that caps and hats, such as baseball caps, are very ubiquitous and comfortable to wear. The individual would have already adjusted the cap 16 so that a proper and comfortable fit would be attained. The present invention takes advantage of this fact by attaching its device to the cap 16, as will be more fully described herein.

A eye shield 18, which may be in form of a lens such as a corrective vision lens or sunglass lens or the like, is attached to the sleeve 12. As illustrated in FIG. 1, a base member 20 is attached to the sleeve 12, such as by adhesive or the like. A connector 22 is removably attached to the base 20, such as by snap-fit connection or the like. The connector 22 is configured to support and hold the lens 18. In a particularly preferred embodiment the connector 22 includes an open-faced slot 24 into which an upper edge of the lens 18 can be inserted. Thus, the lens 18 can be removed from the device 10 by removing the lens from the connector 22, or disengaging the connection between the connector 22 and base 20. Such may be desirable, for example, when switching lenses 18, such as when switching from a vision correcting lens to a sunglass lens or the like. The removability of the lens 18 is also advantageous when the wearer (not shown) does not require the lens 18 to be in front of his or her eyes. As discussed above, the removal of sunglasses is often necessary when walking into a building or driving through a tunnel or the like. The wearer of the device 10 could simply detach the lens 18 from either the connector 22, or the connector 22 and lens 18 from the base 20 in such situations.

With reference now to FIGS. 2-4 and 12-15, a more preferred method of attaching the lens 18 to the sleeve 12 or bill 14 is by means of a hinge assembly 26 comprising the base 20, connector 22 and an articulating body 28 connected therebetween. As illustrated, the articulated body 28 includes an upper connection member 30 which is removably attachable to the base 20, such as being inserted into an open-faced groove 32 and having projections 34 which extend through apertures 36 of the base 20. The upper connection member 30 is pivotally connected to a body 38 by virtue of a hinge 40. The body 38 is connected to a lower connection member 42 by a second hinge 44. The lower connection member 42 is detachably connected to the connector 22 in a manner similar to that described above with the connector 22 having an open-faced slot 46 which removably receives the lower connection member 42. For added connection, the lower connection member 42 may include protrusions 48 which are received within apertures 50 of the connector 22.

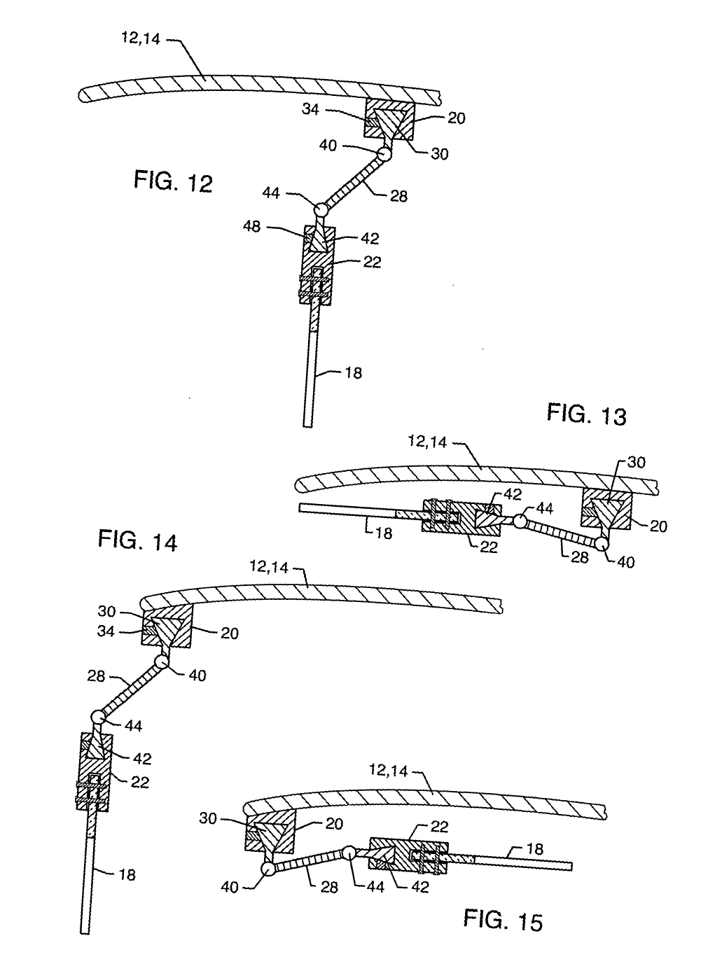

With reference now to FIGS. 12-15, the hinge assembly 26 enables a wearer of the device 10 of the present invention to pivot the lens or other eye shield 18 relative to the eyes of the wearer such that they are aligned with the wearer's eyes, as illustrated in FIG. 12 and 14, or folded upwardly towards the sleeve 12 or bill 14 of the cap, as illustrated in FIGS. 13 and 15, so as to not impede the normal vision of the wearer. Those skilled in the art will appreciate that the dual hinges 40 and 44 of the articulating body 28 enable the lens 18 to be positioned at many angles so as to accommodate different wearer's anatomy and size, as well as to assist the wearer in controlling the placement of the lens 18 as the need arises. FIGS. 12 and 13 illustrate the attachment of the base 20 closer to the forehead of the wearer, while the base 20 is attached to the sleeve 12 or bill 14 away from the wearer's forehead, depending upon the job performed or the desires of the wearer of the device 10.

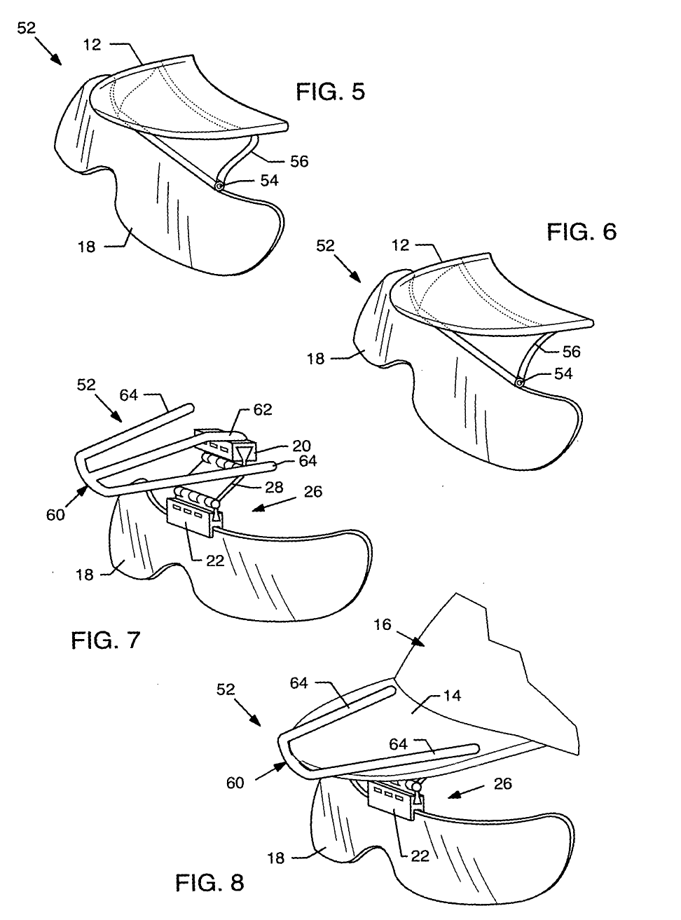

With reference now to FIGS. 5 and 6, another device 52 is illustrated wherein the lens 18 is pivotally connected to the sleeve 12 by means of a hinge 54, or other pivotal connection between the lens 18 and an extension body 56. The extension body is fixed or otherwise attached to the sleeve 12 and has a contour such that the lens 18 is disposed approximately in alignment with the wearer's eyes. The lens or eye shield 18 can be pivoted upwardly and out of view of the wearer, or in other positions which accommodate the needs of the wearer. Similar to the embodiment discussed above, the sleeve 12 is slipped over a bill 14 of a cap 16, such as a baseball cap or the like having a bill protruding therefrom.

With references now to FIGS. 7 and 8, yet another device 58 embodying the present invention is illustrated which instead of a sleeve 12 incorporates the use of a clip 60 for attaching the hinge assembly 26 and lens or eye shield 18 to the bill 14 of the cap 16. In a particularly preferred embodiment as illustrated, at least one elongated arm 62 of the clip 60 is attached to the base 20. At least one other elongated arm 64, and preferably multiple elongated arms 64 are disposed generally parallel to the lower elongated arm 62 such that they can be disposed over the top surface of the bill 14, while the one or more lower elongated arms 62 are disposed under the bill 14 so as to retain the bill 14 between the lower and upper arms 62 and 64, as illustrated in FIG. 8.

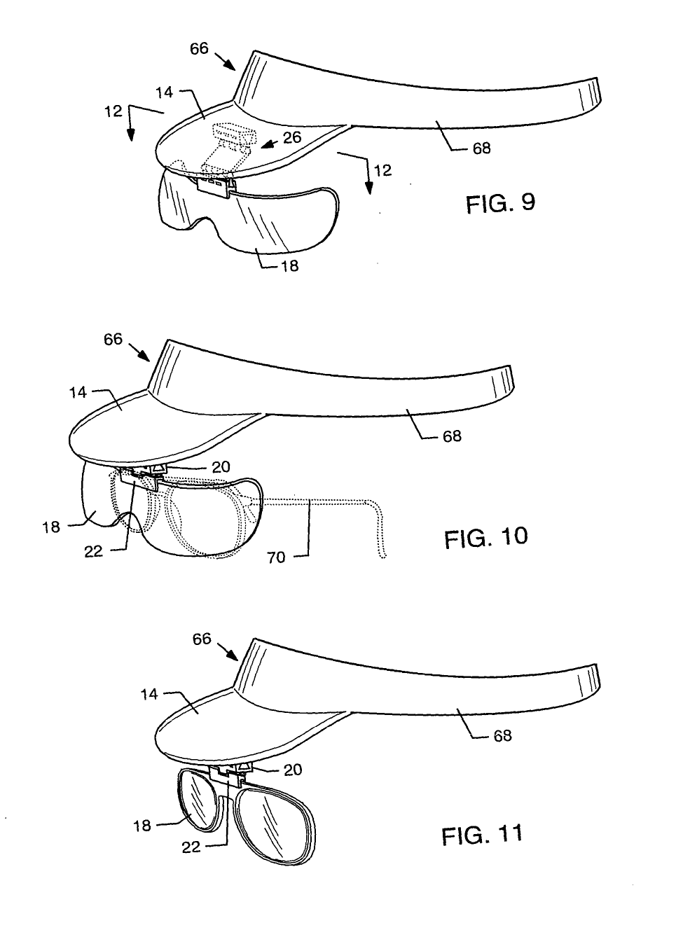

With reference now to FIGS. 9-11, yet another embodiment of the device 66 is illustrated as a visor comprising a headband 68 and attached bill 14, in normal fashion. The eye shield or lens 18 may be pivotally attached to the bill 14 using the hinge assembly 26, or other pertinent structure, as illustrated in FIG. 9. Alternatively, the eye shield or lens 18 may be simply detachably connected to the bill 14 by utilizing such structure as the connector 22 and base 20 as described above. Preferably, the eye shield 18 is configured such so as to be usable over normal eyeglasses 70 of a user. Thus, the user could wear his normal vision correcting eyeglasses 70 and also wear the visor 66 with a magnifying lens, sun shading lens, etc. It is contemplated that the lens 18 could also comprise shatter-resistance or shatter-proof material so as to be used as safety eyewear as well. With reference now to FIG. 11, instead of serving as a shield over normal eyeglasses 70, the lens 18 could actually comprise normal corrective vision lenses. Alternatively, the lens 18 could serve the previously described benefits and purposes while being of a different configuration to meet the esthetic needs and desires of the wearer. Conceivably, the headband 66 could be inserted over a baseball cap or the like, or worn on the head of the user in normal fashion.

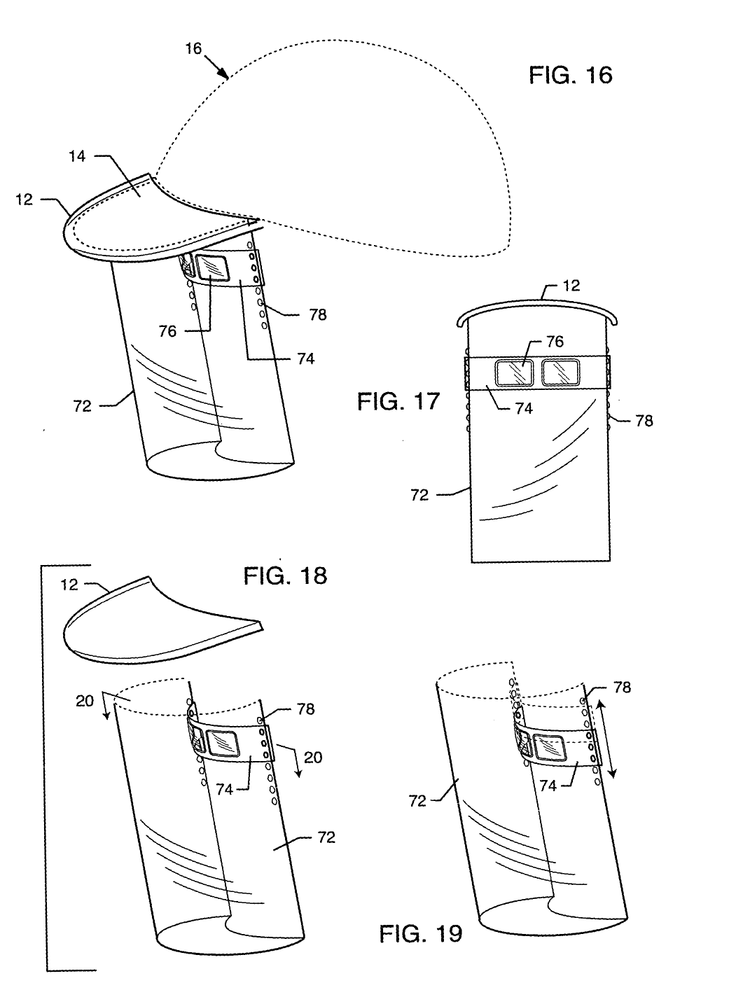

With reference now to FIGS. 16-21, yet another embodiment of the present invention is illustrated wherein a face shield 72 is formed with or otherwise attached to the sleeve 12. Preferably, the face shield 72 has a curve which matches the curve of the sleeve 12 and bill 14 of the cap 16. Such a face shield 72 is preferably sized such so as to shield the wearer's entire face from the exterior environment. Such face shields 72 are particularly useful for medical workers, such as surgeons and dentists and the like. However, the face shield 72 may also be beneficially used in machine shop and the like jobs where dangerous debris is emitted from grinders, cutters, etc. A lens 74 is attached to opposite edges of the face shield 72. The lens 74 may include specialized lenses 76, such as vision correcting lenses or magnifying lenses. Alternatively, the inset lenses 76 would comprise the actual lenses through which the wearer views, with the exterior larger lens 74 serving merely as a connection member.

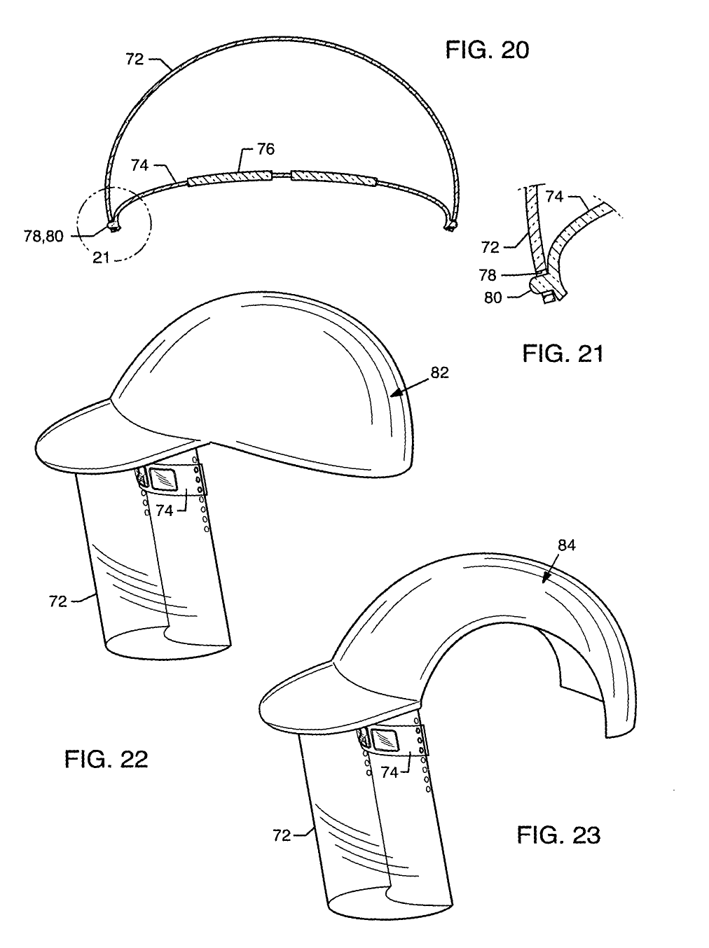

In a particularly preferred embodiment, as illustrated, the lens or lens connecting member 74 is movably positioned along the face shield 72 to accommodate different users. One means of accomplishing this is to provide a series of apertures 78 along opposing edges of the face shield 72. As shown in FIGS. 20 and 21, the lens or lens connecting member 74 includes a mating protrusion 80 which can be snapped-fit into aligned aperture 78 so as to be removably held in place.

With reference now to FIGS. 22 and 23, while the face shield 72 may be positioned using the sleeve 12 discussed above, and illustrated in FIGS. 16-18, the face shield 72 can also be associated with the hat or cap by other means. For example, as illustrated in FIGS. 22 and 23, a shell 82 or 84 may be provided which is fixed to or otherwise formed with the face shield 72 and which is configured to overlie a baseball cap or the like. The shell 82 may be configured so as to completely overlie the exterior surface of the cap, or only partially overlie the cap, as illustrated in FIG. 23. The user achieves the benefit of wearing a comfortable cap or hat while removably associating the lens 74 and face shield 72.

Particularly in the medical setting, such face shields 72 are disposable after a single use, such as a single surgery or the like. Due to the removable nature of the lens assembly 74, the lens assembly 74 can be removed from the disposable face shield 72 and inserted into a new face shield 72 for a subsequent surgery or the like. The face shield 72 may be removably connected to the sleeve 12 or shell 82 and 84, or these mounting members may be disposable as well.

It will be appreciated by those skilled in the art that the embodiments illustrated and described herein provide many benefits which are not found in the prior art. The present invention provides eye protection or vision correction or assistance without the uncomfortable pressure on the bridge of the nose or temples of the wearer. The weight of the devices is supported by the comfortable cap, visor or hat. The eyewear of the present invention provides the user with panoramic vision which is not obstructed in any manner. The user of the invention can replace lenses or shields as necessary to meet different needs, such as a dentist or jeweler using a magnifying lens while at work, yet replacing this lens with a sun shield for driving. Instead of having to completely remove the head gear, the wearer of the present invention can merely alter the position of the lens or shield in many of the embodiments by pivoting the lens or shield upwardly or down to reposition the lens either out of view of the wearer's eyes, or in alignment with the wearer's eyes. The present invention also accommodates the users normal eyeglasses if necessary.

Although several embodiments have been described in detail for purposes of illustration, various modifications may be made without departing from the scope and spirit of the invention. Accordingly, the invention is not to be limited, except as by the appended claims.

1. A device associated with a cap having a bill, which allows a wearer to selectively view objects through a lens, comprising:
a mounting member adapted to be removably attached to the bill of the cap; and
an eye shield including the lens pivotally attached to the mounting member so as to be selectively positionable relative to eyes of the wearer

2. The device of claim 1, wherein the mounting member comprises a sleeve adapted to removably receive a bill of the cap therein.

3. The device of claim 1, wherein the mounting member comprises a clip for removable attachment with the bill of the cap.

4. The device of claim 3, wherein the clip comprises a plurality of elongated arms, at least one elongated arm being positionable above the bill, and at least one elongated arm being positionable below the bill so as to retain the bill between the upper and lower elongated arms.

5. The device of claim 1, including a hinge assembly interconnecting the mounting member and the eye shield.

6. The device of claim 5, wherein the hinge assembly comprises a base attached to the mounting member, an articulating body attached at a first end thereof to the base and at a second end thereof to an eye shield connector.

7. The device of claim 6, wherein the articulating body is removably attached to the base.

8. The device of claim 6, wherein the connector is removably attached to the articulating body.

9. A device which allows a wearer to selectively view objects through a lens, comprising:
a visor having a bill; and
an eye shield including the lens removably attached to the bill of the visor so as to be postionable relative to eyes of the wearer.

10. The device of claim 9, including a base attached to the bill and a eye shield connector connected to the base and the eye shield.

11. The device of claim 10, wherein the connector is removably connected to the base and the eye shield.

12. The device of claim 10, including an articulating body attached at a first end thereof to the base and at a second end thereof to the eye shield connector.

13. A device associated with a cap having a bill, which allows a wearer to selectively view objects through a lens, comprising
a mounting member adapted to be removably attached to the cap; and
a face shield attached to the mounting member and supporting the lens thereon such that the lens is positionable relative to eyes of the wearer.

14. The device of claim 13, wherein the mounting member comprises a sleeve adapted to removably receive a bill of the cap therein.

15. The device of claim 13, wherein the mounting member comprises a shell adapted to overlie at least a portion of an exterior surface of the cap.

16. The device of claim 13, wherein the lens is removably attached to the face shield.

17. The device of claim 13, wherein the lens is positionable along at least a length of the face shield to be positioned relative to the wearer's eyes.

18. The device of claim 17, wherein the face shield includes a series of generally aligned apertures which removably receive mating protrusions of the lens by snap-fit connection.

19. The device of claim 13, wherein the lens comprises a vision correction lens.

20. The device of claim 13, wherein the lens comprises an eye shielding lens.
